SEQUENCE LISTING (27 sequences)

SEQ ID NO: 1 - RNA, 25 nt, Hepatitis C virus
acccggaauugccaggacgaccggg

SEQ ID NO: 2 - DNA, 25 bp, artificial
  siDNA against HCV-RNA
cccggtcgtcctggcaattccgggt

SEQ ID NO: 3 - DNA, 25 bp, artificial
  siDNA against HCV-RNA
tgggccttatgccagctcgaaaagg

SEQ ID NO: 4 - RNA, 25 nt, Hepatitis C virus
cccgggaggucucguagacgugcac

SEQ ID NO: 5 - DNA, 25 bp, artificial
  siDNA against HCV-RNA
gtgcacgtctacgagacctcccggg

SEQ ID NO: 6 - DNA, 25 bp, artificial
  siDNA against HCV-RNA
gggccctcctctcgttcacgcacac

SEQ ID NO: 7 - DNA, 54 bp, artificial
  siDNA against HCV-RNA
tgggccttatgccagctcgaaaaggttttcccggtcgtcctggcaattccgggt

SEQ ID NO: 8 - DNA, 54 bp, artificial
  siDNA against HCV-RNA
gggccctcctctcgttcacgcacacttttgtgcacgtctacgagacctcccggg

SEQ ID NO: 9 - DNA, 390 bp, Artificial
  5′-NTR of HCV Replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacc

SEQ ID NO: 10 - RNA, 25 nt, Hepatitis C virus
guaccacgugccagaugcucuggag

SEQ ID NO: 11 - DNA, 54 bp, Artificial
  siDNA against HCV-RNA
ctccagagcatctggcacgtggtacttttgtattacggcccagatcgagacctc

SEQ ID NO: 12 - DNA, 25 bp, Artificial
  siDNA against HCV-RNA
ctccagagcatctggcacgtggtac

SEQ ID NO: 13 - DNA, 21 bp, Artificial
  siRNA against HCV-RNA
ttacgugccagaugcucugga

SEQ ID NO: 14 - DNA, 21 bp, Artificial
  siRNA against HCV-RNA
ttuccagagcaucuggcacgu

SEQ ID NO: 15 - DNA, 54 bp, Artificial
  siDNA against HCV-RNA
cggtatcaccagacgccttggccacttttgtgctcaacccgtctgcactatggc

SEQ ID NO: 16 - DNA, 54 bp, Artificial
  siDNA against HCV-RNA
ttttcttttcccccctgaccttcccttttgggttggtttggggggttttctttt

SEQ ID NO: 17 - DNA, 54 bp, Artificial
  siDNA against HCV-RNA
ttttttagttgcgttgcctgcccgctttttcctttcctccttcttggggggttt

SEQ ID NO: 18 - DNA, 54 bp, Artificial
  siDNA against HCV-RNA
ttttgtactttctcatacccctcctttttaggtcggggttgagaatcgtttttt

SEQ ID NO: 19 - DNA, 25 bp, Artificial
  antisense DNA against HCV-RNA
cggtatcaccagacgccttggccac

SEQ ID NO: 20 - DNA, 54 bp, Artificial
  siDNA against HCV-RNA
gtggccttaacggtcctgctggcccttttgggttagctcagccgtattccggtg

SEQ ID NO: 21 - DNA, 25 bp, Artificial
  siDNA against HCV-RNA
gtggccttaacggtcctgctggccc

SEQ ID NO: 22 - DNA, 54 bp, Artificial
  siDNA against HCV-RNA
gccttaacggtcctgctggcccaggttttcctccgcctccaggacgcaattccg

SEQ ID NO: 23 - DNA, 25 bp, Artificial
  antisense DNA against HCV-RNA
gccttaacggtcctgctggcccagg

SEQ ID NO: 24 - DNA, 54 bp, Artificial
  siDNA against HCV-RNA
cccacgaacgctcacggggccctccttttggaccgccttgtgagccaagcaccc

SEQ ID NO: 25 - DNA, 25 bp, Artificial
  antisense DNA against HCV-RNA
cccacgaacgctcacggggccctcc

SEQ ID NO: 26 - DNA, 54 bp, Artificial
  siDNA against HCV-RNA
gggccctccagagcatctgcacgtgttttcaccggcacttgctctcctcccggg

SEQ ID NO: 27 - DNA, 25 bp, Artificial
  antisense DNA against HCV-RNA
gggccctccagagcatctgcacgtg

(19) United States
(12) Patent Application Publication
(10) Pub. No.: US 2010/0204302 A1
(43) Pub. Date: Aug. 12, 2010

(54) SIDNA AGAINST HEPATITIS C VIRUS (HCV)

(75) Inventor: Karin Moelling, Berlin (DE)

Correspondence Address: JOYCE VON NATZMER;PEQUIGNOT + MYERS LLC, 200 Madison Avenue, Suite 1901, New York, NY, 10016, US

(73) Assignee: UNIVERSITAET ZUERICH, Zuerich (CH)

(21) Appl. No.: 12/676,211

(22) PCT Filed: Aug. 22, 2008
(86) PCT No.: PCT/EP08/07109

§ 371 Date: Mar. 3, 2010

(30) Foreign Application Priority Data
Sep. 3, 2007 (EP) 07075750.5

(51) Int. Cl. A61K 31/713; C07H 21/04; A61P 31/14

(52) U.S. Cl. 514/44 A; 536/23.72

(57) ABSTRACT

Silencing of HCV RNA can be achieved by siDNA. These are oligodeoxynucleotides consisting of an antisense strand homologous to the viral RNA and a second strand, partially complementary to the antisense-strand. The two strands are preferentially linked by a linker (eg 4 thymidines). Triple-helix formation is a preferred effect. The siDNA is superior to siRNA because the formation of RNA-DNA hybrids is preferred over double-stranded DNA or double-stranded RNA, which forms as tertiary structures in RNA genomes. Also the induction of interferon is less likely. siDNA is easier to synthesize and it is more stable. It can be combined with siRNA.

The invention refers to silencing of HCV RNA which can be achieved by siDNA. These are oligodeoxynucleotides consisting of an antisense-strand homologous to the viral RNA and a second strand, partially complementary to the antisense-strand. The two strands are preferentially linked by a linker (eg 4 thymidines). Triple-helix formation is a preferred effect. The siDNA is superior to siRNA because the formation of RNA-DNA hybrids is preferred over double-stranded DNA or double-stranded RNA, which forms as tertiary structures in RNA genomes. Also the induction of interferon is less likely. Furthermore, siDNA is effective at earlier time-points after addition to the cell than siRNA. siDNA is easier to synthesize and it is more stable. It forms RNA-DNA hybrids with the target RNA and thus is more stable than double-stranded RNA in the case of siRNA. It can also be combined with other siDNAs targeted against various strains in a cocktail; it can also be combined with siRNA to target early and late steps in viral replication.

### BACKGROUND OF THE INVENTION

Hepatitis C virus (HCV) is a small (about 50 nm in size), enveloped, single-stranded, positive sense RNA virus in the family Flaviviridae. At least six HCV genotypes are known, which can be further grouped into various subtypes differing in nucleotide sequence by 20 to 25%. Over 170 million people are persistently infected with HCV, and approximately 38′000 new cases are registered annually in the United States alone (CDC Fact sheet). In 50 to 80% of all cases, the virus establishes a persistent infection, often leading to chronic liver disease, such as fibrosis, steatosis, cirrhosis. In 1 to 5% of persons with of patients with chronic infections liver cancer develops over a period of 20 to 30 years. 1 to 5% of infected persons may die. Long term effects are leading indications for liver transplants. Persons at risk for HCV infection might also be at risk for infection with Hepatitis B virus (HBV) or HIV. These infections potentiate the severeness of any single infection. Alcohol also accelerates disease progression.

HCV is transmitted by blood contacts from an infected person to an uninfected one, eg. through needle sharing, needle sticks, exposure to sharp objects for healthcare workers, from an infected mother to her baby during birth.

Current therapy comprises a combination of polyethylene glycol-conjugated alpha (peg) interferon and ribavirin or each component individually. The combination therapy can get rid of the virus in up to 50% for genotype 1 and in up to 80% for genotypes 2 and 3, but success rates are limited, and severe side effects as well as high costs restrict the use of this therapy.

It was recently demonstrated by the inventor the use of siDNA targeted to the HIV genome. This activates the RNase H of the virus and leads to silencing of the viral RNA (Matzen et al, Nature Biotechnol. 25, 669-674 (2007). The RNase H is a Hybrid-specific RNase which cleaves RNA in an RNA-DNA hybrid and was discovered by Moelling et al (Nature New Biology 234, 240-243 (1971)). We also observed that cellular RNase H-like activities such as RNase H1 and RNase H2a,b,c, and even Agonaute 2 contribute to siDNA-mediated silencing. Agonaute 2 (Ago2) is the enzyme known to induce siRNA-mediated silencing. We showed that it Ago2 is enzymatically related to RNases H and can induce siDNA-mediated silencing as well (Moelling et al. Cold Spring Harbor Symposium 71, Small RNAs, (2007)).

Furthermore, it could be shown by the inventor that siDNA is able to induce silencing of an oncogenic retrovirus, the Spleen Focus-Forming Virus and prevent infection, cause delay of disease progression and lead to increased survival time (Nature Biotechnol. 25, 669-674 (2007)). Furthermore the inventor showed in several cases that siDNA is superior to a single-stranded antisense effect (Jendis et al. AIDS Research and Human Retroviruses 12, 1161-1168 (1996), AIDS Research and Human Retroviruses 14, 999-1005, (1998), Moelling et al, FEBS Letters, 580, 3545-3550 (2006), Matzen et al. Nature Biotechnol. 25, 669-674 (2007)).

Thus the effect of siDNA has been demonstrated in several cases.

Recently siRNA has been described against HCV (Chevalier et al., Mol. Ther. May 15, (2007)). The authors selected several siRNAs, some of them are strongly involved in tertiary RNA structures, which are not favored to be opened up by another RNA strand through siRNA.

The problem to be solved with the present invention is to present an effective antiviral therapeutic against Hepatitis C virus (HCV) infections.

The problem of the invention is solved by the features of the independent claims.

An object of the invention is therefore to present siDNA oligonucleotides, capable of binding to one or more RNA target regions of Hepatitis C virus (HCV), as antiviral therapeutic, whereby the siDNA oligonucleotides are consisting of an antisense-strand fully or almost fully homologous to the viral RNA target and a second strand, partially complementary to the antisense-strand forming a partially double stranded hairpin-loop-structured oligonucleotide, comprising G-clusters consisting of at least two G nucleotides in succession to allow tetrade or tetramer formation or tetra-helices or higher-ordered structures within the same siDNA oligonucleotide molecule (cis conformation) or through interaction with another siDNA oligonucleotide molecule (trans conformation). The invention is described below in more detail.

Another object of the invention is to provide a method, respectively the use of siDNA oligonucleotides, or a combination of siDNAs, as antiviral therapeutic for the treatment of Hepatitis C virus (HCV) infections capable of binding to RNA target regions of HCV or different HCV variants, whereby different siDNA oligonucleotides may be combined in a cocktail as pharmaceutical agent.

A further object of the invention is to provide a pharmaceutical composition as antiviral therapeutic for the treatment of Hepatitis C virus (HCV) infections, comprising said siDNA oligonucleotides capable of binding to RNA target regions of HCV as antiviral therapeutic.

### SUMMARY OF THE INVENTION

Therefore, siDNA oligonucleotides, capable of binding to one or more RNA target regions of Hepatitis C virus (HCV), as antiviral therapeutic, are object of the invention whereby the siDNA oligonucleotides are consisting of an antisense-strand homologous to the viral RNA target and a second strand, partially complementary to the antisense-strand forming a hairpin-loop structure. The siDNA oligonucleotides correspond to one or more HCV target regions of at least 20 nucleotides in length, whereby the antisense strand of the siDNA is more than 80%, more preferably more than 90%. homologous to the target HCV-RNA. Further, the second strand of the siDNA oligonucleotides is connected to the antisense strand through a thymidine linker, preferred—but not exclusively—4 nucleotides in length, and whereby further the second strand is partially (40-60% homology) complementary to the antisense-strand and may be able to form triple helices by non-Watson-Crick base pairing with the viral RNA target strand.

As referred before, an object of the invention is also the use of siDNA as antiviral therapeutic. These are oligodeoxynucleotides consisting of an antisense-strand homologous to the viral RNA and a second strand, partially complementary to the antisense-strand. The siDNA is targeted to a conserved region of HCV, 20 to 25 nucleotides or longer, which is fully or almost fully (more than 80%, more preferably more than 90%) homologous to the target RNA. A second strand is connected to the antisense strand through a Thymidine linker, e.g. 4 nucleotides in length. The second strand is partially complementary (40-60%) to the antisense-strand and may be able to form triple helices by non-Watson-Crick base pairing.

Also a preferred embodiment of the invention is the use of a combination of two or three siDNAs as described herein as antiviral therapeutic capable of binding to RNA target regions of HCV as pharmaceutical agent. Further preferred is the use of a cocktail of different siDNA oligonucleotides to target different HCV variants.

The siDNA will be applied to an infected cell or an infected individual with a transducing agent, whereby the transducing agent is selected from the following group: the virus itself, a replicating HCV particle which carries the siDNA into the cell during the process of infection, a liposome, transmembrane carriers or peptides. Most preferred is a cell transfection by using a lipid-based transfection reagent like Lullaby which is known from siRNA silencing. Other lipid based transducing agents are preferred, too.

The siDNA oligonucleotides according to the invention are preferred as antiviral therapeutic as part of a pharmaceutical composition comprising these siDNA oligonucleotides capable of binding to RNA target regions of HCV.

### MORE DETAILED DESCRIPTION OF THE INVENTION

The invention is based on the cognition that a DNA strand has a thermodynamic preference to form an RNA-DNA hybrid over double-stranded DNA or double-stranded RNA. Thus the invention is based on the not expected result that siDNA is of high preference (in contrast to siRNA) to target RNA by forming RNA-DNA hybrids; siDNA is therefore of advantage and a preferred object of the invention. Furthermore siDNA acts against the newly infecting incoming viral RNA, 2 to 3 days before siRNA. Thus a virus infection can be prevented by siDNA only. In contrast thereto, interferon-induction is a risk for siRNA and reduced or absent for an RNA-DNA hybrid or double-stranded DNA. Furthermore, siDNA is preferred due to the fact that DNA is easier to synthesize and more stable. Also, it can be combined with siRNA. Furthermore, siDNA leads to RNA-DNA hybrids, which are more stable than RNA-RNA generated by siRNA.

The siDNA may also be a chimeric molecule, consisting of ribonucleotides and desoxyribonucleotides. However, the RNase H cleavage site needs to consist of a local RNA-DNA hybrid.

siDNA is also superior to a simple antisense oligodeoxynucleotide, because it is more stable during uptake by the cell and inside the cell and therefore more effective. It acts earlier than antisense DNA, and can target incoming RNA, while antisense DNA targets mRNA (Jekerle, V. et. al. J. Pharm. Sci. 8, 516-527 (2005)).

It was shown before that siDNA is targeting viral RNA before replication while siRNA is effective only late during replication targeting the mRNA (Westerhout, E. M. et. al., Retrovirology 3, 57-65 (2006)).

The invention takes advantage of this effect in silencing of HCV RNA. Using siDNA oligonucleotides is significantly more effective in contrast to siRNA. An unexpected advantage in conjunction with the present invention of siDNA versus siRNA is the targeting of incoming viral RNA before replication while siRNA is effective only late during replication targeting the mRNA. At this stage the RNA is highly amplified (e.g. 1000 fold) while the incoming RNA consists of a single copy. Viral RNA is destroyed within 8 to 14 hours post infection by 10 fold reduction with no siRNA effect. At this timepoint the siRNA effect is undetectable. The siRNA needs to enter a multi-protein complex, RISC, to prepare the cleavage, a process which takes about 3 days. By then the virus has replicated efficiently. After 20 to 30 hours the reduction of viral RNA is similar in both cases, amounting to about 3- to 4 fold reduction.

The RNA silencing inside the cell is achieved by the cellular RNases H, such as RNase H1 or RNase H2a,b,c. Also the Ago2, the siRNA-inducing silencing enzyme, is RNase H-like. These enzymes are located mainly in the nucleus but also in the cytoplasm. They are mainly responsible for removing RNA primers during DNA replication in the cell. The siDNA will lead to silencing of the viral RNA during early stages of replication and/or mRNA during late stages of replication, which requires higher doses. Treatment is performed by injection of siDNA into the blood stream or intraperitoneally. Depending on the stability of the compound the treatment can be repeated. Toxicity has not been detected in mouse studies (Matzen et al., Nature Biotech. 25, 669-674 (2007)).

As stated above, it could be shown by the inventor that siDNA is able to induce silencing of an oncogenic retrovirus, the Spleen Focus-Forming Virus and prevent infection, cause delay of disease progression and lead to increased survival time (Nature Biotechnology, Jun. 3, 2007). The mechanism was due to the action of the viral RNase H. A contribution of cellular RNases H or related cellular enzymes is likely. New unpublished results show that a reporter plasmid can be targeted by an siDNA and that the gene expression is silenced by RNase H1 or RNase H2a. This was demonstrated by inactivation of these RNases H, which strongly reduces the silencing effects.

Preferred embodiments of the invention are described following:

Under the term “homology” the following should be understood: A siDNA oligonucleotide according to the invention is consisting of an antisense-strand which is more than 80% homologous to the viral RNA target and a second strand, which is partially complementary to the antisense-strand forming a partially double stranded hairpin-loop-structure. The term “partially complementary” means a homology between 40 and 60% within the hairpin-loop.

Under the term “G-cluster” the following should be understood:

“G-cluster” according to the invention are motifs within a sequence of a siDNA oligonucleotide. A G-cluster is defined by at least 2 G nucleotides in succession (“GG”). Several G-clusters lead to tetrade or tetramer formation or tetra-helices or higher-ordered structures within the siDNA oligonucleotide molecule. Each G-cluster is separated from another G-Cluster by 1 to 20 X nucleotides, wherein X is A, T, C or a single G, followed by A, T or C. General examples (among others) according to said definition are

| | |
|---|---|
| i. | GG |
| ii. | GGG |
| iii. | GGXG |
| iv. | GGGG |
| v. | GGXXXGGXXXXGG |
| vi. | GGXXGGXXXXGGG |

G-tetrade formation is beneficial for stability, cellular uptake, reduced need for carriers consisting e.g. of GGXXGGXXXXGGG etc, whereby X represents basically other nucleotides than “GG”, such as A, T or C or a single G, as long as the single G is followed by A, T or C. The siDNA oligonucleotides according to the invention contain G clusters to allow tetrade or tetramer formation or tetra-helices by those sequences. The hairpin-loop structure may consist preferably of up to 9 non-self-complementary sequences at the 3′- and 5′-ends, followed by 6 base-pairing, 2 non-pairing, 3 pairing, 2 non-pairing, 3 pairing sequences, followed by 4Ts (see for proof of principle Moelling et al, FEBS Letters, 580, 3545-3550 (2006)). A homology of 40 to 60% is preferred.

The tetrade or tetramer formation or tetra-helices or higher-ordered structures within the siDNA oligonucleotide molecule results of an interaction of G-clusters within the same siDNA oligonucleotide molecule (so-called cis conformation) or through interaction with another siDNA oligonucleotide molecule (so-called trans conformation). Both conformations (cis or trans) are possible.

Specific examples according to the invention are (framed sequence motifs are able to tetrade or tetramer formation or tetra-helices or higher-ordered structures; bold nucleotides indicate G-cluster):

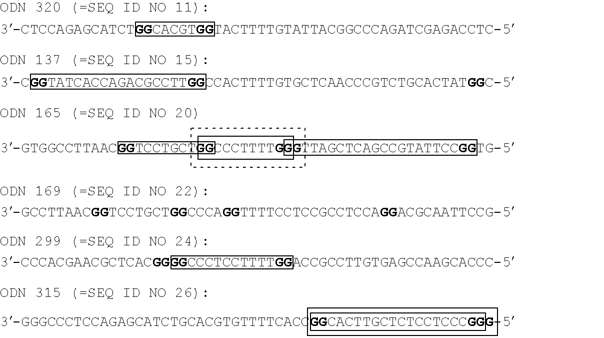

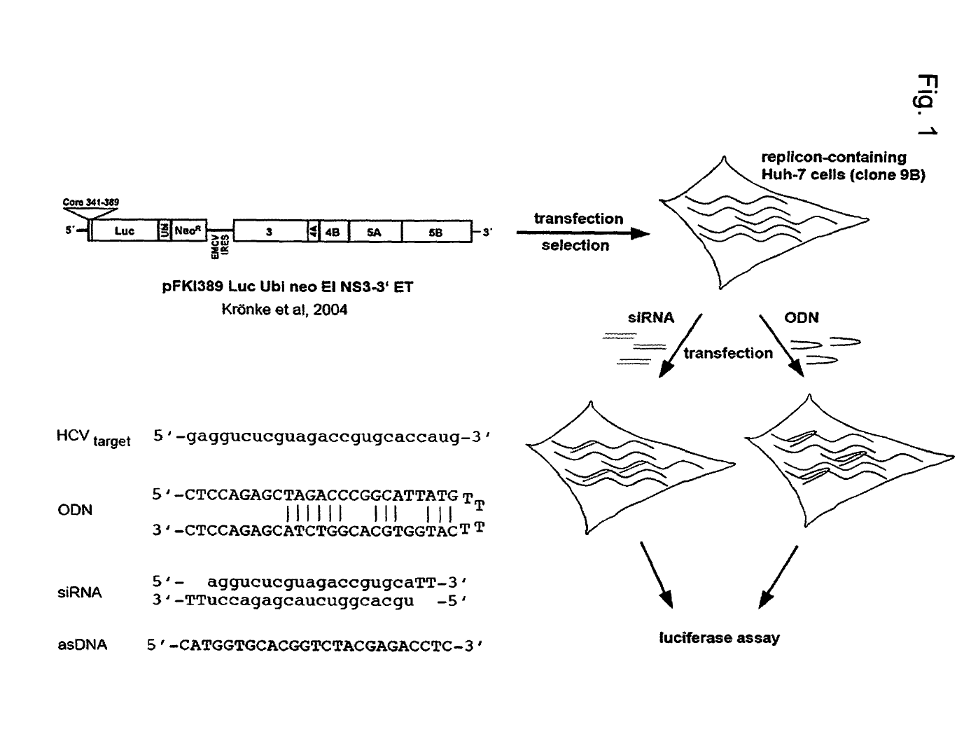

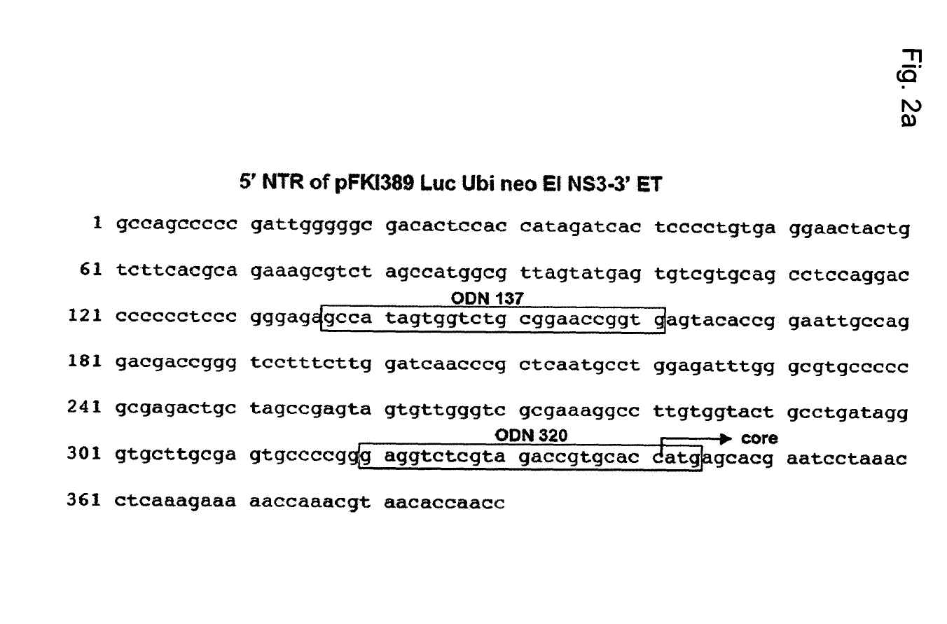

Preferred according to the invention are the sequences for siDNAs as shown below which are also an object of the invention:

| | Sequence | | |
|---|---|---|---|
| Sequence No. | Name | short description | Homology |
| SEQ ID NO 1 | RNA-HCV | Hepatitis C virus | 25/25 |
| | target A | isolate TN24 5′UTR, | (100%) |
| | | partial sequence; | |
| | | ACCESSION No. | |
| | | AF463475 | |
| SES ID NO 2 | siDNA | | 100% |
| | antisense A | | |
| SEQ ID NO 3 | siDNA second | | 50% |
| | strand A | | |
| SEQ ID NO 4 | RNA-HCV | Hepatitis C virus | 20/25 |
| | target B | subtype 6a; | (=80%) |
| | | Accession No. | |
| | | DQ480524 | |
| SEQ ID NO 5 | siDNA | | |
| | antisense B | | |
| SEQ ID NO 6 | siDNA second | | |
| | strand B | | |
| SEQ ID NO 7 | siDNA (HCV) | complete sequence | |
| | | SEQ ID NO 2 and | |
| | | SEQ ID NO 3 | |
| SEQ ID NO 8 | siDNA (HCV) | complete sequence | |
| | | SEQ ID NO 5 and | |
| | | SEQ ID NO 6 | |
| SEQ ID NO 9 | 5′ NTR of | Localization of | |
| | pFKI389 Luc | applied HCV sequence- | |
| | Ubi neo EI | specific ODNs | |
| | NS3-3′ ET | | |
| SEQ ID NO 10 | HCV 320-344 | | |
| SEQ ID NO 11 | ODN 320 | s. FIG. 1 | 100%* |
| SEQ D NO 12 | asDNA 320 | | |
| SEQ D NO 13 | siRNA 320 (1) | | |
| SEQ ID NO 14 | siRNA 320 (2) | | |
| SEQ ID NO 15 | ODN 137 | s. FIG. 1 | 100%* |
| SEQ ID NO 16 | ODN A | | |
| SEQ ID NO 17 | ODN sc | | |
| SEQ ID NO 18 | ODN infl | | |
| SEQ ID NO 19 | asDNA 137 | | |
| SEQ ID NO 20 | ODN 165 | | 100%* |
| SEQ ID NO 21 | asDNA 165 | | |
| SEQ ID NO 22 | ODN 169 | | 100%* |
| SEQ ID NO 23 | asDNA 169 | | |
| SEQ ID NO 24 | ODN 299 | | 100%* |
| SEQ D NO 25 | asDNA 299 | | |
| SEQ ID NO 26 | ODN 315 | | 100%* |
| SEQ D NO 27 | asDNA 315 | | |

*only the Antisense strand

The siDNA “antisense” strands and the siDNA “second strand” are linked via a thymidine (T4) linker.

The siDNA may be stabilized by base modifications e.g. thioates at the ends and/or in the center. The invention is not restricted to those kinds of base modifications. Other base modifications, i.e phosphorothioates, P-DNA, sugar-phopsphate modifications, Morpholinos, amidates, 2′-OMe, 2′-F, 2′-MOE, LNA and further are also preferred without limitation to those modifications.

According to the invention in particular siDNA oligonucleotides comprising at least one sequence selected from the Group: SEQ ID NO 7, SEQ ID NO 8, SEQ ID NO 11, SEQ ID NO 15, SEQ ID NO 20, SEQ ID NO 22, SEQ ID NO 24 or SEQ ID NO 26 are preferred for the use as antiviral therapeutic for the treatment of Hepatitis C virus (HCV) infections and for a corresponding pharmaceutical composition.

Similar to siRNA a combination of two or three siDNAs may be targeted to different regions of the RNA genome or a cocktail may be designed to target different HCV variants. The preferred siDNA sequences are connected by a linker, preferably a thymidine linker, in particular a T4-linker.

siDNA can also be targeted to cellular mRNAs coding for proteins essential for HIV replication and indirectly prevent HCV replication.

The invention shows that an HCV-sequence ODN reduced HCV RNA replication in a sequence-specific and concentration dependent manner. It is preferred according to the invention to use siDNA oligonucleotides in concentrations of about 150 nM±50 nM.

### Materials and Methods

Cells. Human hepatoma cell-line Huh-7 clone 9b containing the subgenomic HCV replicon 1389/NS3-3′/LucUbiNeo-ET (Krönke et al., 2004) was maintained in Dulbecco's Modified Eagle's Medium (Invitrogen) supplemented with 10% fetal calf serum (Brunschwig), 100 U/ml penicillin, 100 μg/ml streptomycin (Invitrogen) and 0.5 mg/ml of G418 (Life Technologies).

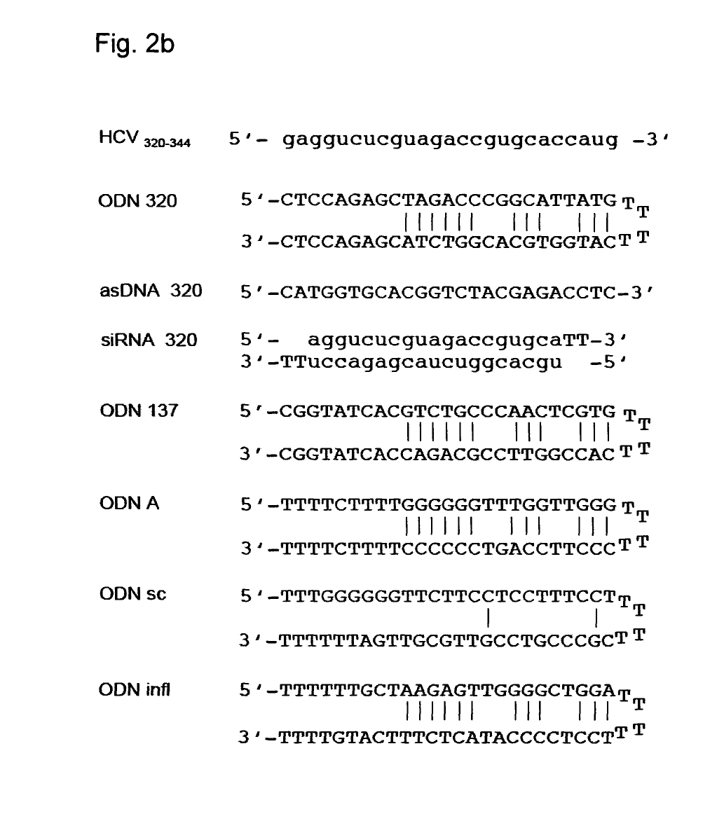

Oligonucleotides. The sequences of all oligonucleotides used in this study are shown in FIG. 2b. Double-stranded oligonucleotides (dsODNs) are hairpin-loop-structures consisting of a 25-mer antisense strand and a partially complementary 25-mer sense strand connected by 4 deoxythymidines. The three bases at the 5′ and 3′ end as well as the linker of four dTTPs are modified by phosphorothioates. Antisense oligodeoxynucleotides (asDNAs) consist of a single-stranded 25-mer modified by phosphorothioates at the three bases at the 5′ and 3′ end (Integrated DNA Technologies). Small interfering RNAs (siRNAs) consist of a 21-mer sense and a 21-mer antisense strand converted to the 2′hydroxyl annealed duplex (Dharmacon RNA Technologies).

Cell transfections. 24 h before transfection cells were resuspended in antibiotics-free DMEM supplemented with 10% FCS and transferred into 12-well plates in a volume of 1000 μl per well. Shortly before the transfection, 700 μl of the culture medium were removed from each well to obtain after addition of the transfection mixture of 200 μl a final volume of 500 μl per well for the transfection periode. 6 h after transfection the medium was replaced by 1 ml DMEM supplemented with penicillin/streptomycin and 10% FCS. Transfections with Lullaby (OZ Biosciences) were carried out according to the manufacturer's recommendations. All transfection mixtures were prepared in serum- and antibiotics-free OptiMEM (Invitrogen).

Luciferase assays. All firefly luciferase activities were measured using the Dual-Luciferase Reporter Assay System (Promega) according to the manufacturer's recommendations. Briefly, cells were washed once with PBS and lysed in 250 μl passive lysis buffer. 20 μl of the cell lysate were used for analysis.

Protein determination. Level of total protein in cell lysates were determined by Bio-Rad Protein Assay (Bio-Rad GmbH). 15 to 25 μl of cell lysate were added to 1000 μl of Bradford reagent solution (diluted 1:5) and OD at 595 nm was measured.

### MORE DETAILED DESCRIPTION OF THE INVENTION, EXAMPLES AND FIGURE DESCRIPTION

In recent studies, it has been shown that small interfering RNA (siRNA) and short hairpin RNA (shRNA) targeting highly conserved sequence motifs within the 5′ non-translated region (5′NTR) efficiently suppress the replication of subgenomic and genomic HCV replicons (see above). A different group of oligonucleotides, referred as partially double-stranded, hairpin-loopstructured oligodeoxynucleotides (dsODN) was used to target HIV-1 RNA. There the viral RNA was shown to be reduced by the action of the HIV-specific RNase H, a virion-associated RNA-DNA hybrid-specific ribonuclease. This enzyme is linked to the Reverse Transcriptase in retroviruses. It is furthermore a virion-associated enzyme activity.

The invention demonstrates that influenza viral RNA can also be reduced by the action of a short partially influenza-specific ODNs in cell culture as well as in a mouse model. The silencing effect of one of the ODNs was similar to the efficincies of antisense DNA and siRNA.

Furthermore, an object of the invention are G-rich regions as sequences of high efficiency for silencing of RNA by ODNs. They can form higher-ordered structures such as G tetrads which are preferred sequences for HVC.

Here, we studied the ability of dsODNs to inhibit HCV RNA replication. As test system we used a subgenomic replicon in which the 3′ part of the core gene and the whole envelope gene were replaced with firefly luciferase as reporter gene and the neomycin phosphotransferase gene for selection (Krönke et al., 2004, J Virol. 78, 3436-46; FIG. 1). The examples below show that in this system an HCV-sequence ODN reduced HCV RNA replication in a sequence-specific and concentration dependent manner. A direct comparison with siRNA and antisense single-stranded DNA to the same sequence was performed and indicates similar efficiencies. This indicates that double-stranded ODNs are an alternative approach for gene silencing, whereby each of the three approaches is distinct and ahs its specific advantage.

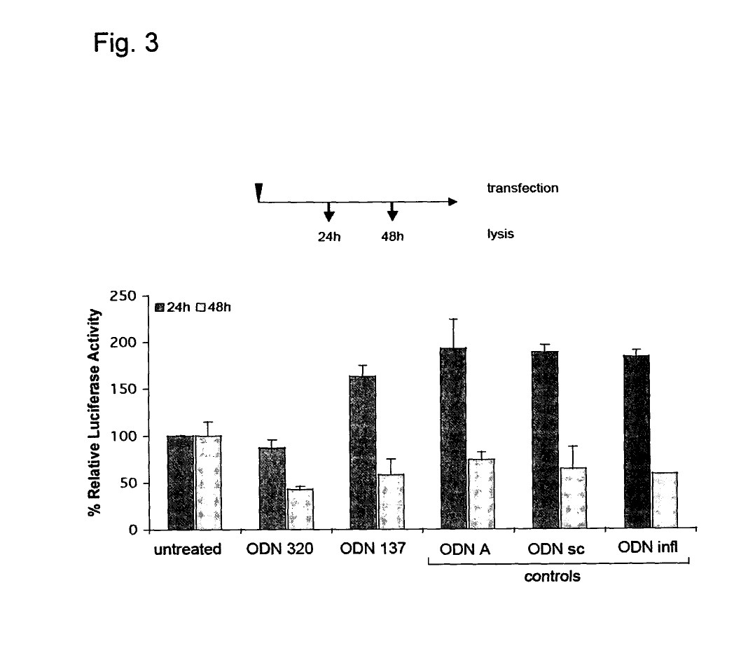

In order to study the effect of dsODNs (dsODN=double stranded ODN) on HCV RNA replication we used the human liver cell-line Huh-7 clone 9B stably expressing the subgenomic replicon 1389/NS3-3′/LucUbiNeo-ET encoding the firefly luciferase as reporter (Krönke et al. 2004), shown in FIG. 1. In a first set of experiments we transfected Huh-7 clone 9B cells with two HCV-sequence specific dsODNs, ODN 320 and ODN 137, and compared them with HVC-unrelated dsODNs ODN A, ODN sc and ODN infl (used previously against inflenca RNA), as control (FIGS. 2 A and B). Cells were lysed 24 h or 48 h after transfection and luciferase reporter activity correlating with HCV RNA expression was measured (FIG. 3). Transfection of ODN 320 reduced the reporter activity to 85% and 40% of the level in untreated control cells within 24 and 48 h, respectively. HCV-unrelated dsODNs increased the reporter activity within 24 h for unknown reasons and decreased it to 75% to 60% of the level of untreated control cells within 48 h. Thus, ODN 320 suppressed HCV RNA replication more efficiently than control ODNs. The effects of the HCV-sequence specific ODN 137 was similar to the HCV-unrelated ODN infl, indicating that it reduces reporter activity not in an HCV-specific manner. It may be unable to interact with its respective target sequence because of the highly structured NTR targeted here.

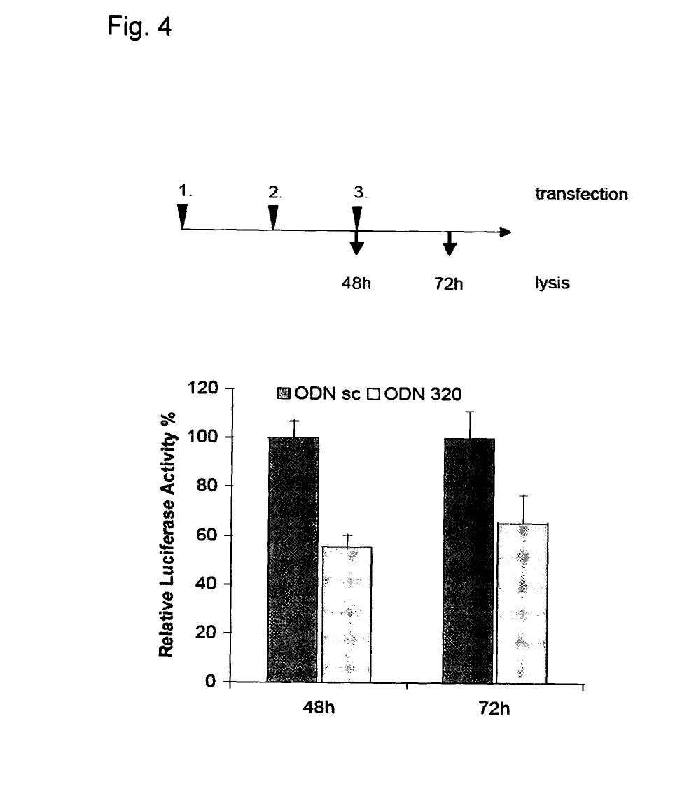

To analyse long-term effects of ODN 320, we analysed the cells 72 h after transfection. These conditions did not further decrease the reporter activity by transfection of ODN 320 compared to the control ODN sc (FIG. 4).

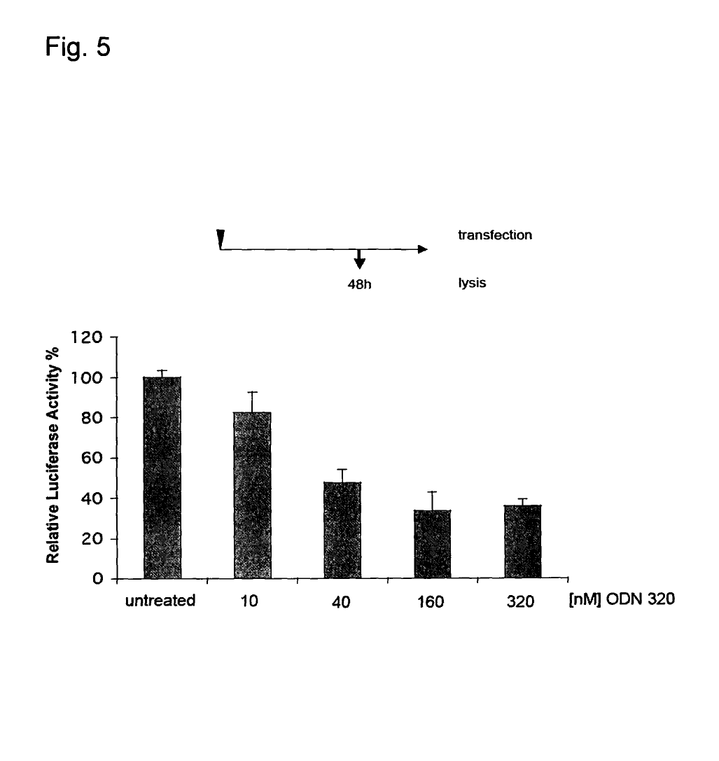

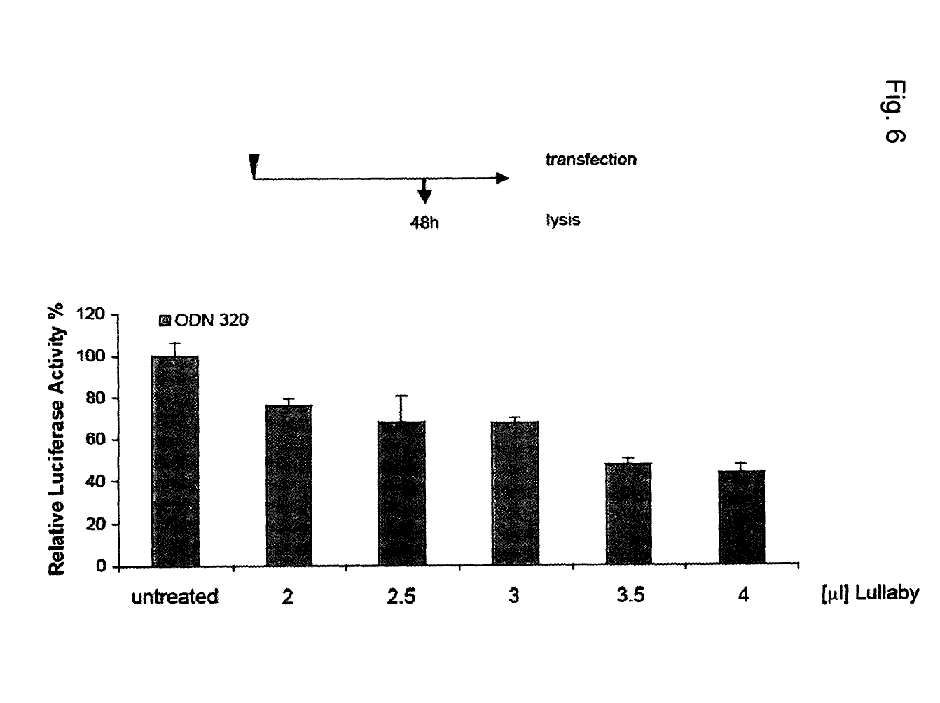

In general, the transfection efficiency of oligonucleotides depends on the ratio of the oligonucleotide to the transfection reagent. To optimize the effect of ODN 320 on suppression of HCV RNA replication we first transfected increasing amounts of ODN 320 with a constant amount of transfection reagent (FIG. 5). The maximal inhibition of reporter activity was 35% of the level of untreated cells and was achieved at a concentration of 100 to 200 nM. In a second set of experiment we varied the amount of transfection reagent at constant amount of ODN 320. At a concentration of 100 nM ODN 320 with 3.5 to 4 μl transfection reagent in 100 μl was optimal and inhibited the reporter activity to 40% (FIG. 6). Further experiments were performed with 3.5 μl transfection reagent and 100 nM ODN.

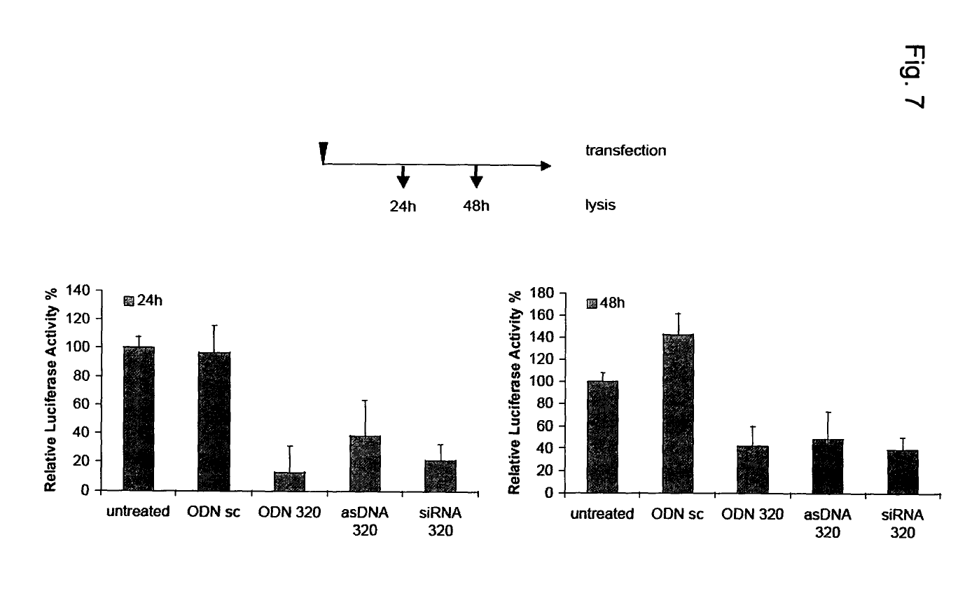

In several studies it has been shown that HCV RNA replication is susceptible to small interfering RNA, siRNA, and single-standed antisense DNA, asDNA. There-fore we compared ODN 320 with siRNA and asDNA targeting the same region of the HCV RNA genome (see FIG. 2b). In this experimental setting ODN 320 inhibited reporter activity to 15% of the level of untreated cells within 24 h. siRNA and asDNA reduced the reporter activity to 20% and 40%, respectively (FIG. 7). 48 h after transfection the effect of ODN 320 was not as pronounced as after 24 h and converged to the effects of siRNA and asDNA (FIG. 7). These results indicate that the silencing effect of ODN 320 on HCV RNA replication was superior to the other two reagents at earlier time points and after later time points of similar efficiency.

As a summary it may be stated that different approaches to inhibit HCV gene expression and replication by oligonucleotides exist including antisense oligodeoxynucleotides and siRNAs (reviewed in Pan Q W, Henry S D, Scholte B J, Tilanus H W, Janssen H L, van der Laan U., 2007. New therapeutic opportunities for hepatitis C based on small RNA. World J. Gastroenterol. 13, 4431-6). The invention shows clearly that a short partially double stranded ODN significantly reduced replication of subgenomic HCV replicons. Oligonucleotides according to the invention, such as ODN 320, targets a sequence in the conserved 5′NTR of HCV suggesting that it may act against different HCV genotypes. Under the conditions tested ODN 320 reduced HCV levels by 75 to 80% of the level of untreated cells. The silencing effect of the ODN appeared at earlier time-points than siRNA, which may be due to the fact, that the unwinding of the so-called RISC complex has been reported to require time, while the siDNA may be accessible to cellular RNases H more readily. The antisense effect was at both time points shown here less effective than the ODN. This may be explained by the fact, that antisense DNA is single-stranded and more accessible to degradation or other effects. In all three silencing effects other mechanisms have not been excludes, such as translational arrest. Thus the mechanisms are complex.

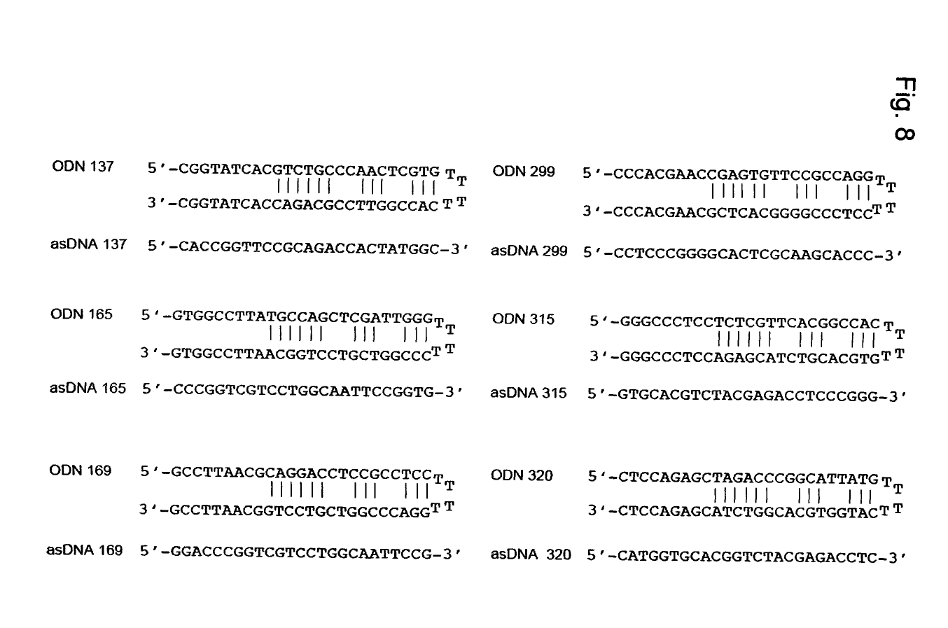

We observed, that G-rich sequences are very effective as shown in the case of HIV (Matzen et al, Nature Biotechnol. 25, 669-674 (2007). A similar region exists in HCV in the E2 gene and this is an important region for targeting (see FIG. 8).

### LEGENDS TO THE FIGURES

FIG. 1: Schematic representation of the subgenomic HCV replicon 1389/NS3-clone 9B. The replicon consists of the HCV 5′ NTR; nucleotides 342-389 of the core coding region (core) fused to the coding sequences of the firefly luciferase gene (Luc), the ubiquitin gene (Ubi), and the neomycin phosphotransferase gene (NeoR); the IRES of encephalomyocarditis virus; the coding region of the HCV nonstructural proteins NS3 to NS5B; and the HCV 3′ NTR. ODNs and siRNAs were transiently transfected into replicon-containing Huh-7 cells of clone 9B. Effects on replication of the subgenomic HCV replicon were analysed by luciferase reporter assays. Silencing by oligonucleotides is detected by reduction of luciferase activity due to RNA degradation of the gene.

The oligonucleotides are indicated as small letters in the case of RNA and capital letters in the case of DNA, abbreviated as ODN. The ODNs used were modified by phosphothioate modifications of three bases at each termini and in the central region in the T4 linker.

FIG. 2: Oligonucleotides used in this study. (a) Sequence of the 5′ NTR of HCV. The HCV sequence-specific ODNs are marked. (b) List of HCV sequence-specific and control oligonucleotides used in this study. ODN A targets HIV-1 (Jendis et al., 1996), ODN infl targets influenza A (Moelling et al. unpublished), ODN sc represents a scrambled sequence of ODN A.

FIG. 3: Effect of HCV sequence-specific ODNs on the replication of the subgenomic HCV replicon. Huh-7 clone 9B cells resuspended in 500 μl medium were seeded into 12-well plates (0.6×105 cells per well and 0.4×105 per well for the time points 24 h and 48 h, respectively) and transfected with the indicated ODNs at a concentration of 160 nM (4 μl Lullaby). Cells were harvested 24 h or 48 h after transfection as indicated and luciferase activity was determined. Values of the luciferase activity were corrected for the relative amount of protein of each sample. This example shows that HCV sequence-specific ODN 320 reduced HCV replication compared to control ODNs.

FIG. 4: Effect of multiple transfections with ODNs. 0.4×105 cells were seeded per well and transfected with ODNs at a concentration of 100 nM (3.5 μl Lullaby) 2 or 3 times in intervals of 24 h. Cells were lysed 48 h and 72 h after the first transfection and luciferase activity was determined as described in FIG. 3. This example shows that multiple transfections did not improve the inhibitory effect of ODN 320 on HCV replication

FIG. 5: Effect of ODN concentration on replication of the subgenomic HCV replicon. Huh-7 clone 9B cells were transfected with different amounts of the HCV sequence-specific ODN 320 (final concentration 10 to 500 nM) and a constant amount of the transfection reagent Lullaby (4 μl). Luciferase activities were determined 48 h after transfection of the cells. At constant amount of Lullaby (4 ml/500 ml) the optimal concentration of ODN 320 is 100-200 nM.

FIG. 6: Effect of Lullaby concentration on replication of the subgenomic HCV replicon. Huh-7 clone 9B cells were transfected with different amounts of the transfection reagent Lullaby and a constant amount of ODN 320 (final concentration 100 nM). Luciferase activities were determined 48 h after transfection of the cells. At a concentration of 100 nM ODN 320 the optimal amount of Lullaby is 3.5-4 ml/500 ml.

FIG. 7: Effects of ODN, antisense DNA and siRNA on replication of the subgenomic HCV replicon. Huh-7 clone 9B cells were transfected with oligonucleotides as indicated at a concentration of 100 nM (3.5 μl Lullaby). Luciferase activities were determined 24 h and 48 h after transfection of the cells. ODN 320 inhibited HCV replication at least as good as antisense DNA and siRNA.

FIG. 8: Other ODN sequences targeted to the 5′NTR of HCV. The numbers indicate the 1st nucleotide shown in FIG. 2a.

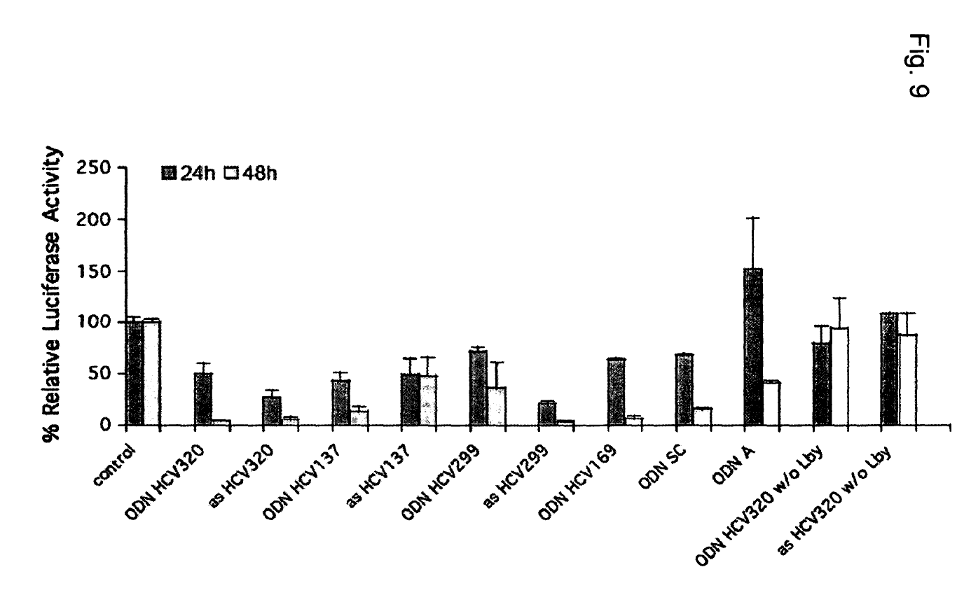

FIG. 9: Preliminary results obtained with additional ODNs indicated in FIG. 8. ODN 320 was less effective without Lullaby than in its presence.

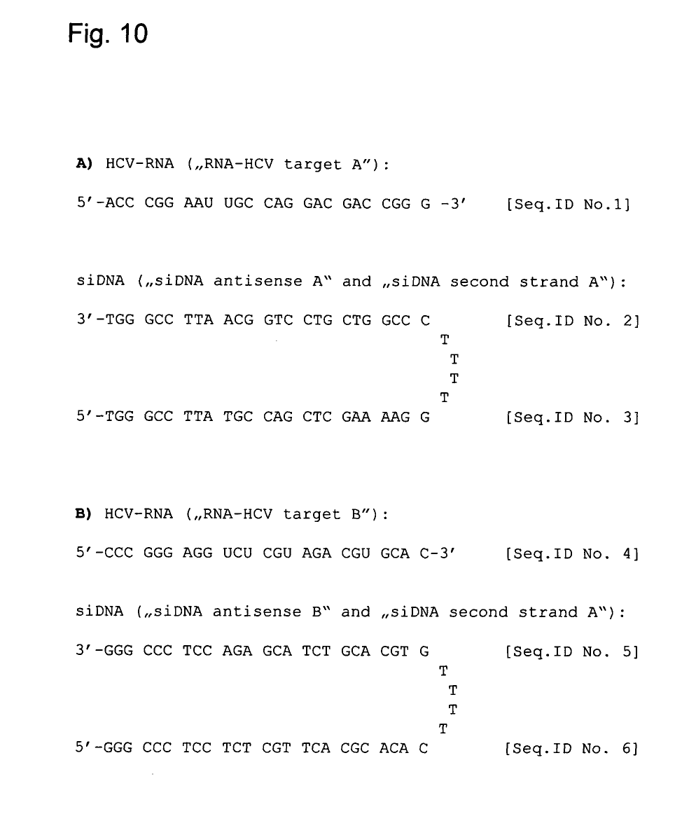

FIG. 10. Sequences according to SEQ ID NO 1 to SEQ ID NO 8.

1. Small interfering DNA (siDNA) oligonucleotides, capable of binding to one or more RNA target regions of Hepatitis C virus (HCV), as antiviral therapeutic, wherein each of said siDNA oligonucleotides comprises
an antisense-strand fully or almost fully homologous to the one or more RNA target regions of HCV and
a second strand, partially complementary to the antisense-strand forming a partially double stranded hairpin-loop-structured oligonucleotide, comprising
G-clusters of at least two G nucleotides in succession to allow tetrade or tetramer formation or tetra-helices or higher-ordered structures within an siDNA oligonucleotide molecule (cis conformation) or through interaction with another siDNA oligonucleotide molecule (trans conformation).

2. The siDNA oligonucleotides according to claim 1, wherein the siDNA molecule corresponds to one or more HCV target regions of at least 20 nucleotides in length, wherein the antisense strand of the siDNA is more than 80%, more preferably more than 90%, homologous to the target HCV-RNA.

3. The siDNA oligonucleotides according to claim 1, wherein the second strand is connected to the antisense strand through a thymidine linker, preferred 4 nucleotides in length, and wherein the second strand is, within the hairpin-loop, with a homology of 40 to 60%, partially complementary to the antisense-strand and is able to form triple helices by non-Watson-Crick base pairing with the viral RNA target strand.

4. The siDNA oligonucleotides according to claim 2, wherein at least two the G-clusters are separated from each other by other nucleotides.

5. The siDNA oligonucleotides according to claim 1, wherein the siDNA oligonucleotides are stabilized by base modifications.

6. The siDNA oligonucleotide according to claim 1, selected from the group of sequences consisting of SEQ ID NO 7, SEQ ID NO 8, SEQ ID NO 11, SEQ ID NO 15, SEQ ID NO 20, SEQ ID NO 22, SEQ ID NO 24 and SEQ ID NO 26.

7. Method of treating Hepatitis C virus (HCV) infections comprising
administering to a subject in need thereof, in a HCV infection treating effective amount, siDNA oligonucleotides, or a combination of siDNAs oligonucleotides according to claim 1, as antiviral therapeutic for treating Hepatitis C virus (HCV) infections capable of binding to RNA target regions of HCV or different HCV variants, wherein different siDNA oligonucleotides are optionally combined in a cocktail as a pharmaceutical agent.

8. The method of claim 7, wherein the siDNA will be oligonucleotides are applied to an infected cell or an infected individual with a transducing agent.

9. The method of claim 8, wherein the transducing agent is selected from the following group: the virus itself, a replicating HCV particle which carries the siDNA into the cell during the process of infection, a liposome, transmembrane carriers or peptides.

10. The method of claim 7, wherein said siDNA oligonucleotides are used administered in concentrations of about 150 nM±50 nM.

11. Pharmaceutical composition as antiviral therapeutic for the treatment of Hepatitis C virus (HCV) infections, comprising siDNA oligonucleotides according to claim 1 capable of binding to RNA target regions of HCV as antiviral therapeutic.

12. The pharmaceutical composition according to claim 11, comprising at least one sequence selected from the Group: SEQ ID NO 7, SEQ ID NO 8, SEQ ID NO 11, SEQ ID NO 15, SEQ ID NO 20, SEQ ID NO 22, SEQ ID NO 24 or SEQ ID NO 26.

13. The siDNA oligonucleotide according to claim 4, wherein the nucleotides are A, T, C or G.
